(19) United States
(12) Patent Application Publication
(10) Pub. No.: US 2019/0180000 A1
(43) Pub. Date: Jun. 13, 2019

(54) PATIENT DIAGNOSIS AND TREATMENT BASED ON GENOMIC TENSOR MOTIFS

(71) Applicant: INTERNATIONAL BUSINESS MACHINES CORPORATION, Armonk, NY (US)

(72) Inventors: Filippo Utro, Pleasantville, NY (US); Kahn Rhrissorrakrai, Woodside, NY (US); Laxmi Parida, Mohegan Lake, NY (US); Aldo Guzman Saenz, Yorktown Heights, NY (US)

(21) Appl. No.: 15/834,660

(22) Filed: Dec. 7, 2017

(51) Int. Cl. G06F 19/18; G16H 20/10; G06F 19/24

(52) U.S. Cl. CPC G06F 19/18; G06F 19/24; G16H 20/10

(57) ABSTRACT

Methods and systems for genetic diagnosis include splitting genomes into respective groups of non-overlapping windows. The genomes are sampled into sets, each set being made up of selected genomes. A distribution of events is generated across the sets in each window. A tensor is determined for each window based on statistical properties of the distribution of events for the window. A classifier is generated based on the tensors. One or more phenotypes is diagnosed from an input genome using the classifier.

### BACKGROUND

### Technical Field

The present invention generally relates to genomic analysis and, more particularly, to the extraction of information from a set of distinct phenotypes to determine correlations between genomic variations and phenotypical expressions.

### Description of the Related Art

Determining the genomic basis of particular traits involves determining correlations between a person's genotype (the particular sequence that makes up the person's genetic code) and the person's phenotype (the expression of the genotype in traits). However, these correlations can be subtle and difficult to discover, with multiple gene sequences playing a role in the expression of certain phenotypes. This complexity is particularly significant when it comes to identifying diseases and other disorders, both within a specific person and across entire populations.

### SUMMARY

A genetic diagnosis method includes splitting genomes into respective groups of non-overlapping windows. The genomes are sampled into sets, each set being made up of selected genomes. A distribution of events is generated across the sets in each window. A tensor is determined for each window based on statistical properties of the distribution of events for the window. A classifier is generated based on the tensors. One or more phenotypes is diagnosed from an input genome using the classifier.

A genetic diagnosis method includes splitting genomes into respective groups of non-overlapping windows. The genomes are sampled into a plurality of sets, each set being made up of selected genomes with repetition allowed. A distribution of events across the plurality of sets is determined in each window by counting a number of events within the window for each of the sets. A tensor is determined for each window based on statistical properties of the distribution of events for the window by forming an n-tuple from a mean, a variance, a skewness, and a kurtosis of the distribution of events. A classifier is generated based on the tensors. One or more phenotypes are diagnosed from an input genome using the classifier. A treatment is automatically administered to an individual based the diagnosis.

A system for genetic diagnosis includes a gene sequence module configured to split genomes into respective groups of non-overlapping windows. A sampling module is configured to sample the genomes into a plurality of sets, each set being made up of selected genomes. A tensor module includes a processor configured to determine a distribution of events across the plurality of sets in each window and to determine a tensor for each window based on statistical properties of the distribution of events for the window. A training module is configured to generate a classifier based on the tensors. A diagnosis module is configured to diagnose one or more phenotypes from an input genome using the classifier.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is a block diagram illustrating tensor motif based diagnosis and treatment of genetic conditions in accordance with an embodiment of the present invention;

FIG. 2 is a block/flow diagram illustrating the training of a genetic classifier based on a tensor that describes event distribution across genomes in accordance with an embodiment of the present invention;

FIG. 3 is a block/flow diagram illustrating feature selection based on tensors that describe event distribution across genomes in accordance with an embodiment of the present invention;

FIG. 4 is a diagram illustrating an exemplary distribution of events across genomes in accordance with an embodiment of the present invention;

FIG. 5 is a block diagram of a motif-based genetic diagnosis and treatment system in accordance with an embodiment of the present invention; and

FIG. 6 is a block diagram of a processing system in accordance with an embodiment of the present invention.

### DETAILED DESCRIPTION

Embodiments of the present invention provide diagnosis and adaptive treatment to individuals based on classification and analysis of individual genomes. The present embodiments create classifiers based on the statistical properties of samples of a group of different genomes and furthermore help localize regions of a genome that contribute to the expression of particular phenotypes (e.g., localizing the portions that contribute to particular disease).

To accomplish this, the present embodiments subdivide individual genomic sequences into windows and generate a distribution of “events” for each such window. A “tensor” is then formed for each window that characterizes the distribution of events. The tensors are then used as input to a machine learning process that, for example, creates a classifier or performs feature selection to determine which portions of the genome are more relevant for a given phenotype.

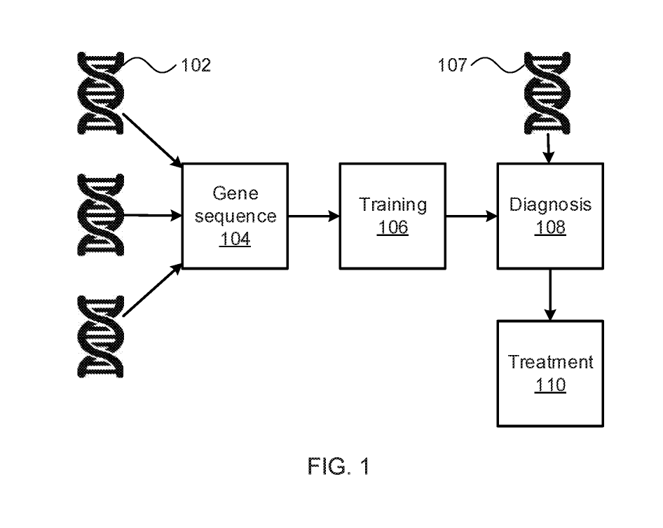

Referring now to FIG. 1, a diagram illustrating the functional relationship of the present embodiments is shown. A set of training genomes 102 are sequenced in gene sequencing 104, breaking the chromosomes in question down into a sequence of individual base pairs and, optionally, whole genes. In this embodiment, the training sequences are used in training block 106 to train a machine learning classifier that can, for example, identify the presence of genetic indicators that lead to the expression of a particular phenotype (e.g., a disease or genetic condition).

Block 108 then performs diagnosis using the genome 107 of an individual under treatment. This diagnosis may include additional factors, such as the individual's medical history, diagnoses by human doctors, lists of symptoms and vitals, and other information relevant to the health of the individual. Block 110 then treats the individual in accordance with the diagnosis. This treatment may involve the intervention of a human medical professional or may, alternatively, be performed automatically through the adjustment of dosages or the administration of drugs. In one specific example, the present embodiments may be employed to distinguish between different kinds of cancer (e.g., breast cancer, lung cancer, ovarian cancer, prostate cancer, etc.) or sub-types of a single kind of cancer. The present embodiments may furthermore differentiate between patients who will be responsive to a given treatment and those patients who will not.

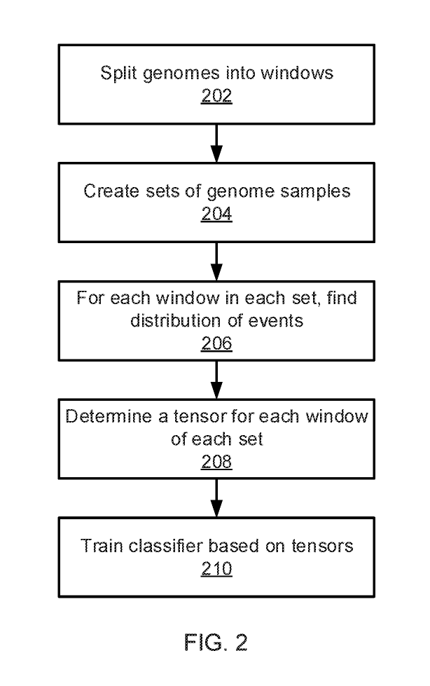

Referring now to FIG. 2, a training method for genetic classifiers is shown. Block 202 divides each genome 102 into a set of non-overlapping windows. In some embodiments, the windows may be for a fixed size (e.g., a predetermined number of base pairs or a certain amount of data such as 50 kB). In other embodiments, the windows may divide certain regions of the genome (e.g., cytobands or other areas of interest) into a predetermined number of windows. The windows may include only gene-coding regions or may include only non-coding regions or both coding and non-coding regions. Furthermore, in embodiments where both coding and non-coding regions are analyzed, the windows may have different sizes in the respective regions. Thus, for each genome g, there will be J windows wⱼ.

Block 204 samples the genomes 102, generating sets sᵢ, each sampling N genomes to form X sets. It should be noted that this sampling may be performed with repetition, such that a given genome may be selected more than once for membership in a given set. The sampling may be performed randomly or may, alternatively, be performed according to any appropriate selection criteria.

Block 206 finds a distribution of events for each window wⱼ in each set sᵢ. The term “event” is used herein to describe any type of genetic feature such as, e.g., a mutation. In some embodiments, block 206 simply counts the number of events in each such window and finds the distribution of event counts across the different sets, though it should be understood that other functions of the number of events can be used instead. Thus, for example, each of ten different sets may have different numbers of events in a given window, and the statistical comparison between the sets provides information regarding the population. Events may include, for example, mutations, copy number variation alteration, gene disruption, and structural variants.

Block 208 determines a tensor for each window wⱼ. In some embodiments, the “tensor” may be a simple n-tuple that encodes particular statistical features. For one specific example, each tensor Tⱼ may be a 4-tuple that includes the mean, variance, skewness, and kurtosis of the distribution relating to a respective window wⱼ across the sets sᵢ. It should be understood that any appropriate statistical information may be used to build the tensors instead.

Block 210 then trains a classifier based on the tensors. In one embodiment, the training is performed by splitting the sets S into two groups, with a first being used to train the classifiers and the second being used to test the classifiers. In particular, many machine learning processes use a training group as input, for example determining a model that recognizes correspondences between the input genotypes and known phenotypes. Machine learning then uses the testing group to test the generated classifier(s), with the genotypes of the testing group being analyzed and used to predict the known phenotypes of that group. Disagreements between predictions and the known results are then used as feedback to the model to correct the model and improve its accuracy. Types of machine learning analyses include, e.g., neural networks, support vector machine processes, linear discriminant analysis processes, random forest processes, and Bayesian processes. Any one of these types of machine learning, or any other variety, may be used to form the classifiers.

The classifiers that are generated may subsequently be used in, for example, diagnosis 108. Taking an individual genome 107 as input, the classifier determines whether the genome in question indicates the likely manifestation of a particular phenotype.

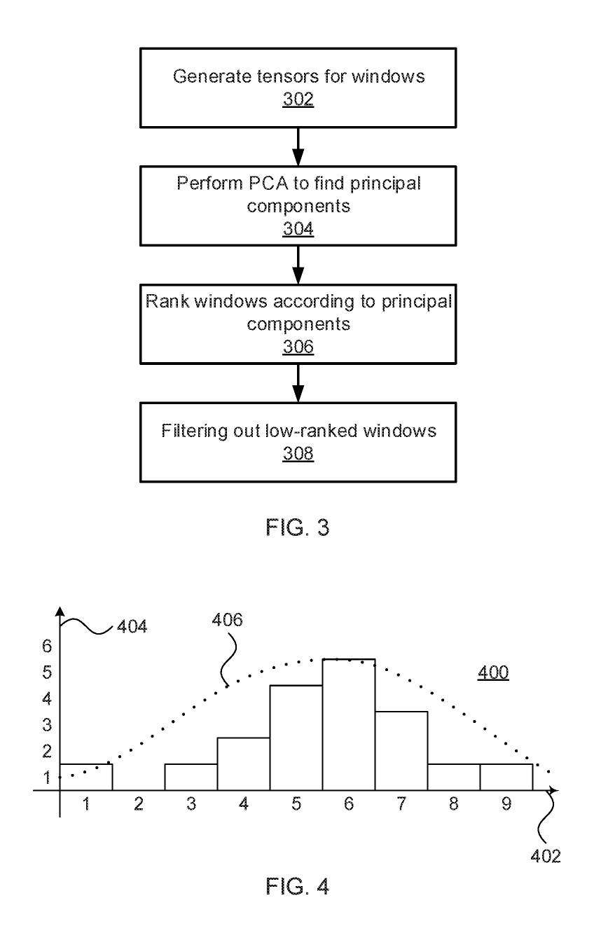

Referring now to FIG. 3, a method of performing feature selection is shown. It should be noted that, although one specific embodiment of feature selection is described herein, other tests such as, e.g., a Kolmogorov-Smirnov test, may be employed instead. Block 302 generates tensors for the windows wⱼ in the sets sᵢ in the same manner as disclosed above with respect to FIG. 2. Block 304 then performs a principal component analysis (PCA) to determine the principal components of the distribution of phenotypes. PCA is a statistical process that uses a transformation to convert a set of data points into a set of uncorrelated variables called the principal components. As a result of this process, it is possible that certain windows will have little influence on the expression of a given phenotype. Block 306 ranks the windows according to the principal components, with windows having little influence on the expressed phenotype being ranked lower than windows having a greater influence. Block 308 then filters out low-ranked windows, for example those windows being below a certain rank or having a contribution below a certain threshold. The threshold value will generally depend on the distribution of high- and low-ranked windows. For example, if there are many features concentrated at low ranks, the threshold should be correspondingly low.

In this manner, feature selection can be performed using the tensor analysis described above. The selected windows can then be used for subsequent analyses, simplifying the analysis by ignoring those windows that provide little contribution to the outcome—depending on the ranking scheme, the rank of a window will provide certain assurances as to what conditions the window may satisfy. The selected windows may be referred to as “motifs,” and they represent the portions of a genome most relevant to the expression of the phenotype in question. These motifs may be used in, for example, a genome-wide association study to help localize the genetic sequences associated with particular traits.

Referring now to FIG. 4, an exemplary event distribution 400 is shown. The vertical axis 404 represents a number of events in a given window and the horizontal axis 402 represents a number of windows having that number of events. In this example, most windows have a number of events between 5 and 7, with outliers to either side.

A statistical distribution 406 is fit to the data and may be in the form of, e.g., a Gaussian curve or any other appropriate distribution. The fit may be performed by any appropriate technique including, for example, a least squares fit. Based on the statistical distribution 406, certain statistical information can be extracted such as, e.g., the mean, the standard deviation, the skew, the kertosis, etc. This information characterizes the relationship between the window in question and the phenotype, with the distribution of events playing a role in how the phenotype manifests across a population.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

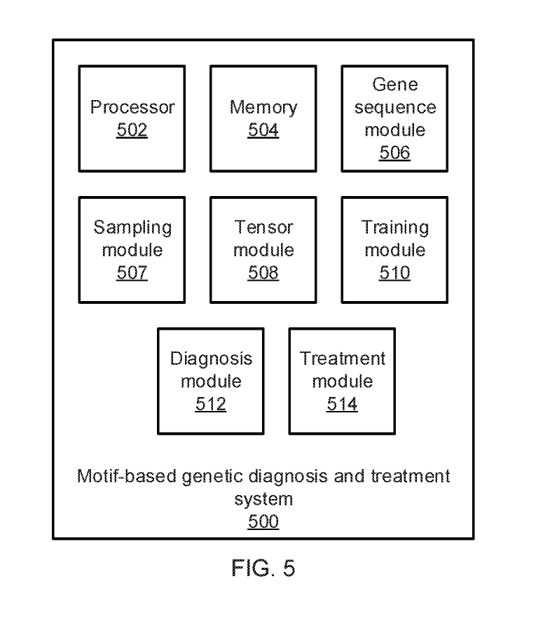

Referring now to FIG. 5, a motif-based genetic diagnosis and treatment system 500 is shown. The system 500 includes a hardware processor 502 and memory 504. The system 500 may also include one or more functional modules that may, in some embodiments, be implemented as software that is stored in the memory 504 and that is executed by the hardware processor 502. In other embodiments, the functional modules may be implemented as one or more dedicated hardware components in the form of, e.g., application-specific integrated chips or field programmable gate arrays. In still other embodiments, the functional modules may be implemented as a piece of dedicated hardware that is controlled by hardware or software logic.

A gene sequence module 506 handles the genomes of individuals. In some embodiments the gene sequence module 506 operates pre-sequenced genomes, while in other embodiments the gene sequence module 506 itself sequences one or more genomes. The gene sequence module 506 splits the input genomes into a set of windows, whether of fixed or variable size.

Sampling module 507 generates sets of samples by selecting genomes from the input genomes, with repetition being allowed in the genomes in any given set. Tensor module 508 then identifies the distribution of events across the sets of genomes on a per-window basis, generating a tensor based on statistical information gleaned from the distribution.

Training module 510 uses one or more machine learning processes to train a classifier and/or to select relevant windows. The training module 510 makes use of a set of training data that is stored in the memory 504, splitting the training data into a training group and a testing group. The training module 510 thereby generates a classifier that identifies whether a given input genome corresponds to a phenotype in question.

Diagnosis module 512 then accepts as input the genome of a specific individual after the input genome has been handled by gene sequence module 506. The diagnosis module 506 determines whether the individual has or is likely to exhibit the phenotype in question (which may include, for example, a disease or subtype of a disease). A treatment module 514 then administers a treatment to the patient, either indirectly (e.g., by providing recommended treatment information to a human medical professional) or directly (e.g., by triggering the automatic administration of drugs). The treatment module 514 may therefore include, or be in communication with, a hardware device configured to administer such a treatment.

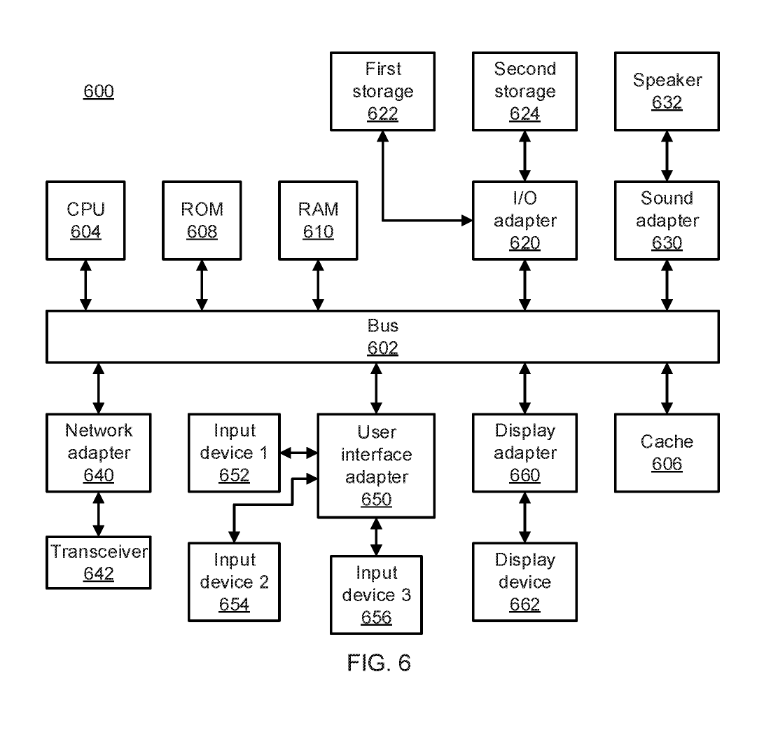

Referring now to FIG. 6, an exemplary processing system 600 is shown which may represent the motif-based genetic diagnosis and treatment system 500. The processing system 600 includes at least one processor (CPU) 604 operatively coupled to other components via a system bus 602. A cache 606, a Read Only Memory (ROM) 608, a Random Access Memory (RAM) 610, an input/output (I/O) adapter 620, a sound adapter 630, a network adapter 640, a user interface adapter 650, and a display adapter 660, are operatively coupled to the system bus 602.

A first storage device 622 and a second storage device 624 are operatively coupled to system bus 602 by the I/O adapter 620. The storage devices 622 and 624 can be any of a disk storage device (e.g., a magnetic or optical disk storage device), a solid state magnetic device, and so forth. The storage devices 622 and 624 can be the same type of storage device or different types of storage devices.

A speaker 632 is operatively coupled to system bus 602 by the sound adapter 630. A transceiver 642 is operatively coupled to system bus 602 by network adapter 640. A display device 662 is operatively coupled to system bus 602 by display adapter 660.

A first user input device 652, a second user input device 654, and a third user input device 656 are operatively coupled to system bus 602 by user interface adapter 650. The user input devices 652, 654, and 656 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input devices 652, 654, and 656 can be the same type of user input device or different types of user input devices. The user input devices 652, 654, and 656 are used to input and output information to and from system 600.

Of course, the processing system 600 may also include other elements (not shown), as readily contemplated by one of skill in the art, as well as omit certain elements. For example, various other input devices and/or output devices can be included in processing system 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 600 are readily contemplated by one of ordinary skill in the art given the teachings of the present principles provided herein.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A genetic diagnosis method, comprising
splitting a plurality of genomes into respective groups of non-overlapping windows;
sampling the plurality of genomes into a plurality of sets, each set comprising a plurality of selected genomes;
determining a distribution of events across the plurality of sets in each window;
determining a tensor for each window based on statistical properties of the distribution of events for the window;
generating a classifier based on the tensors; and
diagnosing one or more phenotypes from an input genome using the classifier.

2. The method of claim 1, wherein sampling the plurality of genomes comprises a sampling with repetition allowed, such that any set may include a given genome more than once.

3. The method of claim 1, wherein determining the distribution of events comprises counting a number of events within the window for each of the sets.

4. The method of claim 1, wherein determining the tensor comprises forming an n-tuple from the statistical properties of the distribution of events.

5. The method of claim 4, wherein the tensor comprises a mean, a variance, a skewness, and a kurtosis of the distribution of events.

6. The method of claim 1, further comprising automatically administering a treatment to an individual based the diagnosis.

7. The method of claim 1, further comprising performing a principal component analysis to rank the windows according to each window's contribution to one or more phenotypes.

8. The method of claim 7, wherein generating the tensor comprises selecting only those windows having a contribution to the one or more phenotypes that is above a threshold value.

9. The method of claim 1, wherein splitting a plurality of genomes into respective groups of non-overlapping windows comprises splitting a corresponding region of each genome into a fixed number of windows.

10. A non-transitory computer readable storage medium comprising a computer readable program for genetic diagnosis, wherein the computer readable program when executed on a computer causes the computer to perform the steps of claim 1.

11. A genetic diagnosis method, comprising
splitting a plurality of genomes into respective groups of non-overlapping windows;
sampling the plurality of genomes into a plurality of sets, each set comprising a plurality of selected genomes with repetition allowed;
determining a distribution of events across the plurality of sets in each window by counting a number of events within the window for each of the sets;
determining a tensor for each window based on statistical properties of the distribution of events for the window by forming an n-tuple from a mean, a variance, a skewness, and a kurtosis of the distribution of events;
generating a classifier based on the tensors;
diagnosing one or more phenotypes from an input genome using the classifier; and
automatically administering a treatment to an individual based the diagnosis.

12. A system for genetic diagnosis, comprising
a gene sequence module configured to split a plurality of genomes into respective groups of non-overlapping windows;
a sampling module configured to sample the plurality of genomes into a plurality of sets, each set comprising a plurality of selected genomes;
a tensor module comprising a processor configured to determine a distribution of events across the plurality of sets in each window and to determine a tensor for each window based on statistical properties of the distribution of events for the window;
a training module configured to generate a classifier based on the tensors; and
a diagnosis module configured to diagnose one or more phenotypes from an input genome using the classifier.

13. The system of claim 12, wherein the sampling module is further configured to sample with repetition allowed, such that any set may include a given genome more than once.

14. The system of claim 12, wherein the tensor module is further configured to count a number of events within the window for each of the sets.

15. The system of claim 12, wherein the tensor module is further configured to form a tensor as an n-tuple from the statistical properties of the distribution of events.

16. The system of claim 15, wherein the n-tuple comprises a mean, a variance, a skewness, and a kurtosis of the distribution of events.

17. The system of claim 12, further comprising a treatment module configured to automatically administer a treatment to an individual based the diagnosis.

18. The system of claim 12, wherein the training module is further configured to perform a principal component analysis to rank the windows according to each window's contribution to one or more phenotypes.

19. The system of claim 18, wherein the tensor module is further configured to select only those windows having a contribution to the one or more phenotypes that is above a threshold value.

20. The system of claim 12, wherein the gene sequence module is further configured to split a corresponding region of each genome into a fixed number of windows.
